(19) United States
(12) Patent Application Publication
(10) Pub. No.: US 2024/0139434 A1
(43) Pub. Date: May 2, 2024

(54) VASCULAR PUNCTURE DEVICE AND VASCULAR PUNCTURE SYSTEM

(71) Applicant: Terumo Kabushiki Kaisha, Tokyo (JP)

(72) Inventors: Takumi FUKUDA, Hadano-shi (JP); Takito INUKAI, Isehara-shi (JP); Yoichiro KUWANO, Tokyo (JP); Yoshinobu ISAKA, Hadano-shi (JP)

(73) Assignee: Terumo Kabushiki Kaisha, Tokyo (JP)

(21) Appl. No.: 18/408,691

(22) Filed: Jan. 10, 2024

Related U.S. Application Data
(63) Continuation of application No. PCT/JP2022/027826, filed on Jul. 15, 2022.

(30) Foreign Application Priority Data
Jul. 21, 2021 (JP) 2021-120219

(51) Int. Cl. A61M 5/42

(52) U.S. Cl. CPC A61M 5/427; A61M 2205/3303; A61M 2210/12

(57) ABSTRACT

A vascular puncture device punctures a blood vessel using an imaging unit that comes into contact with a skin surface and acquires a cross-sectional image of a human body, an inner needle including a sharp needle tip, a flexible outer tube covering the inner needle, a drive unit moving the inner needle and the outer tube, and a detection unit detecting puncture of the needle tip with respect to a blood vessel back wall, and includes a control unit capable of receiving information on the cross-sectional image, controlling an operation of the drive unit, and receiving a detection result from the detection unit. The control unit controls the drive unit to move the inner needle and the outer tube to puncture the blood vessel with the inner needle, and controls the drive unit to stop the puncture with the inner needle.

### CROSS-REFERENCES TO RELATED APPLICATIONS

This application is a continuation of International Application No. PCT/JP2022/027826 filed on Jul. 15, 2022, which claims priority to Japanese Application No. 2021-120219 filed on Jul. 21, 2021, the entire content of both of which is incorporated herein by reference.

### TECHNOLOGICAL FIELD

The present disclosure generally relates to a vascular puncture device and a vascular puncture system capable of detecting and puncturing a position of a blood vessel from an image acquired by an echographic device.

### BACKGROUND DISCUSSION

In order to secure an access route to a blood vessel for drug administration and endovascular treatment, vascular puncture is performed in which a human body is punctured with an inner needle having a sharp needle tip, the inner needle being covered by a flexible outer tube. The access route can be secured due to the outer tube since only the inner needle is removed after the inner needle and the outer tube reach the inside of the blood vessel. In the vascular puncture, an operator cannot visually observe the blood vessel from a skin surface, and thus, a position of the blood vessel is estimated by standard knowledge of blood vessel running and skill such as tactile perception of blood vessel pulsation.

In recent years, there is a device that identifies a position of a blood vessel by a sensor, determines a puncture angle and a puncture path from a shape of the blood vessel or the like, and automatically performs vascular puncture by a robot arm (see, for example, U.S. Pat. No. 9,364,171).

By the way, for example, puncture of a radial artery is currently performed using a method in which a position of a blood vessel is identified based on the visual sense and tactile sense of an operator and a position of a needle tip with respect to the blood vessel is identified based on the presence or absence of backflow of blood from an inner needle. A method of sticking a needle into both a front wall and a back wall of a blood vessel, then retracting the needle, and removing the needle from the back wall, that is, so-called double wall puncture (DWP) is widely used in order to increase a success rate of the puncture and reduce the number of trials. Compared to a method of sticking an inner needle only into a front wall of a blood vessel, that is, so-called single wall puncture (SWP), the DWP has no difference in terms of bleeding and occurrence of radial artery occlusion (RAO).

When the vascular puncture is automatically performed, skill such as tactile perception of an operator cannot be used in order to obtain a state where a distal end of an outer tube is disposed in the blood vessel. Therefore, when the vascular puncture is automatically performed, it is difficult to dispose the distal end of the outer tube in the blood vessel in an appropriate state. For example, in a case where the distal end of the outer tube is not sufficiently inserted into the blood vessel, the distal end of the outer tube may come out of the blood vessel after the inner needle is removed.

### SUMMARY

A vascular puncture device and a vascular puncture system are disclosed, which are capable of appropriately disposing a distal end of an outer tube, which covers an inner needle, inside a blood vessel when vascular puncture is automatically performed.

A vascular puncture device is disclosed that punctures a blood vessel using an imaging unit that comes into contact with a skin surface and acquires a cross-sectional image of a human body, an inner needle including a needle tip that is sharp, an outer tube that is flexible and covers the inner needle, a drive unit that moves the inner needle and the outer tube, and a detection unit that detects entry of a distal end of the outer tube into the blood vessel, or detects contact or puncture of the needle tip with respect to a back wall of the blood vessel, and includes a control unit capable of receiving information on the cross-sectional image, controlling an operation of the drive unit, and receiving a detection result from the detection unit. The control unit controls the drive unit to move the inner needle and the outer tube to puncture the blood vessel with the inner needle, and controls the drive unit to stop the puncture with the inner needle in a case where the detection unit detects the entry of the distal end of the outer tube into the blood vessel or detects the contact or puncture of the needle tip with respect to the back wall of the blood vessel.

A vascular puncture system is disclosed that includes: an imaging unit that comes into contact with a skin surface and acquires a cross-sectional image of a human body; an inner needle including a needle tip that is sharp; an outer tube that is flexible and covers the inner needle; a drive unit that moves the inner needle and the outer tube; a detection unit that detects entry of a distal end of the outer tube into the blood vessel or puncture of the needle tip with respect to a back wall of the blood vessel; and a control unit capable of receiving information on the cross-sectional image, controlling an operation of the drive unit, and receiving a detection result from the detection unit. The control unit controls the drive unit to move the inner needle and the outer tube to puncture the blood vessel with the inner needle, and controls the drive unit to stop the puncture with the inner needle in a case where the detection unit detects the entry of the distal end of the outer tube into the blood vessel or detects the puncture of the needle tip with respect to the back wall of the blood vessel.

The vascular puncture device and the vascular puncture system configured as described above can appropriately dispose the distal end of the outer tube inside the blood vessel regardless of skill of an operator when automatically performing the vascular puncture using the inner needle by stopping the puncture in a case where the entry of the distal end of the outer tube into the blood vessel is detected or stopping the puncture in a case where the puncture of the needle tip into the back wall of the blood vessel is detected.

### BRIEF DESCRIPTION OF THE DRAWINGS

FIG. 1 is a side view of a vascular puncture system according to the present embodiment.

FIG. 2 is a top view of the vascular puncture system, which illustrates a positional relationship with an arm whose cross-sectional image is to be acquired.

FIG. 3 is a configuration diagram of the vascular puncture system.

FIG. 4 is a view illustrating an example of an image acquired by an imaging unit.

FIG. 5 is a side view illustrating the vascular puncture system immediately before puncture in a state where a probe is inclined with respect to a skin surface.

FIG. 6 is a top view illustrating the vascular puncture system immediately before the puncture in the state where the probe is inclined with respect to the skin surface.

FIG. 7 is a side view illustrating the vascular puncture system immediately after the puncture in the state where the probe is inclined with respect to the skin surface.

FIGS. 8A to 8C are schematic views for describing a positional relationship between a blood vessel and a puncture unit, in which FIG. 8A illustrates a state where a front wall is punctured with an inner needle, FIG. 8B illustrates a state where a back wall is punctured with the inner needle, and FIG. 8C illustrates a state where a distal end of an outer tube hits the back wall.

FIG. 9 is a flowchart illustrating a flow of control in a control unit.

FIG. 10 is a flowchart illustrating a flow of control in a first modification.

FIG. 11 is a flowchart illustrating a flow of control in a second modification.

FIG. 12 is a flowchart illustrating a flow of control in a third modification.

FIGS. 13A and 13B are side views illustrating modifications, in which FIG. 13A illustrates a fourth modification, and FIG. 13B illustrates a fifth modification.

FIG. 14 is a cross-sectional view of a puncture unit illustrating a sixth modification.

FIG. 15 is a flowchart illustrating a flow of control in the fourth and fifth modifications.

### DETAILED DESCRIPTION

Set forth below with reference to the accompanying drawings is a detailed description of embodiments of a vascular puncture device and a vascular puncture system capable of detecting and puncturing a position of a blood vessel from an image acquired by an echographic device. Note that dimensional ratios in the drawings may be exaggerated and different from actual ratios for convenience of description.

A vascular puncture system 10 according to the embodiment of the present disclosure is used when puncturing an arm H of a human body to acquire a cross-sectional image of the arm H, detect a position of an artery to be punctured, and automatically puncture the artery.

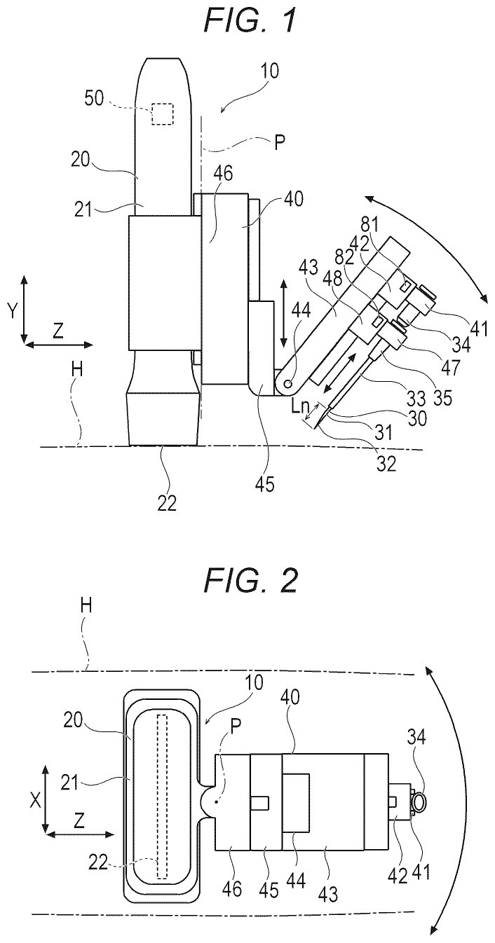

As illustrated in FIGS. 1 to 3, the vascular puncture system 10 includes: a probe 20 having an imaging unit 22 that comes into contact with a skin surface and acquires a cross-sectional image of a human body; a puncture unit 30 that performs puncture; a drive unit 40 that moves the puncture unit 30 with respect to the probe 20; an inclination detection unit 50 that detects an inclination angle of the probe 20; a display unit 70 capable of displaying the cross-sectional image; a detection unit 80 that detects the puncture of a blood vessel by the puncture unit 30 and entry of the puncture unit 30 into the blood vessel; and a vascular puncture device 11. The vascular puncture device 11 includes a control unit 60 that performs image analysis of the cross-sectional image and controls the drive unit 40.

The probe 20 includes a vertically elongated handle portion 21 gripped by an operator, an imaging unit 22 disposed at a lower end of the handle portion 21, a transmitter 23 that transmits a signal from the control unit 60 to the imaging unit 22, and a receiver 24 that transmits a signal from the imaging unit 22 to the control unit 60.

The imaging unit 22 is provided so as to extend over substantially the entire width at the central portion of a lower surface of the probe 20. The imaging unit 22 is an echographic device that includes a transducer that generates an ultrasound wave and obtain the cross-sectional image of the inside of the human body by detecting a reflected wave of the ultrasound wave. In the present embodiment, the cross-sectional image orthogonal to an axial direction of the blood vessel is acquired, and thus, the imaging unit 22 is disposed such that a length direction of the imaging unit 22 is orthogonal to a length direction of the arm H.

The transmitter 23 transmits a signal from the control unit 60 to the imaging unit 22 in order to output an ultrasound wave from the imaging unit 22. The receiver 24 transmits, to the control unit 60, a signal output from the imaging unit 22 receiving the reflected wave.

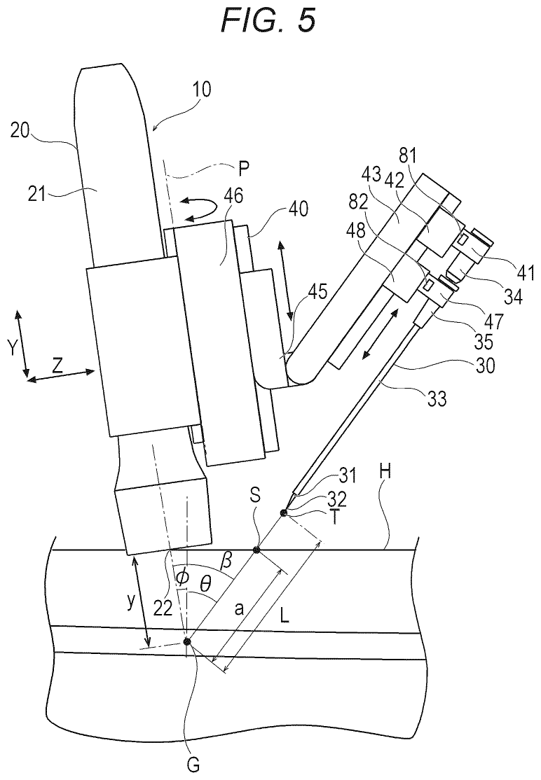

The inclination detection unit 50 is connected to the control unit 60. The inclination detection unit 50 can be, for example, a gyro sensor, and can detect an inclination of the probe 20. A reference of the inclination is a perpendicular direction orthogonal to the horizontal direction. Since an upper surface of the arm H when performing the puncture is set along the horizontal direction, an inclination of the vascular puncture system 10 with respect to a perpendicular line of the skin surface can be detected by detecting the inclination with respect to the perpendicular direction by the inclination detection unit 50. In the present example, it is assumed that the inclination detection unit 50 detects that the vascular puncture system 10 is inclined at an angle of φ as illustrated in FIG. 5. Note that the inclination detection unit 50 is not limited to the gyro sensor, and may be, for example, a camera that captures an image of the skin surface of the arm H. In this case, the control unit 60 can detect the inclination φ of the probe 20 from a result of the image capturing by the inclination detection unit 50 using a technique such as machine learning or deep learning. In addition, the inclination detection unit 50 is not necessarily provided.

As illustrated in FIGS. 1 and 5, the puncture unit 30 includes a solid inner needle 31 made of metal and having a sharp needle tip 32 formed at a distal end, and a flexible tubular outer tube 33 disposed so as to cover an outer peripheral surface of the inner needle 31. Since the inner needle 31 is solid, the outer diameter of the inner needle 31 can be reduced in a case where the inner needle 31 has rigidity similar to that of a hollow needle. Therefore, a puncture hole formed by the inner needle 31 can be reduced, bleeding can be reduced, and burden on a patient can be reduced. The inner needle 31 may be hollow.

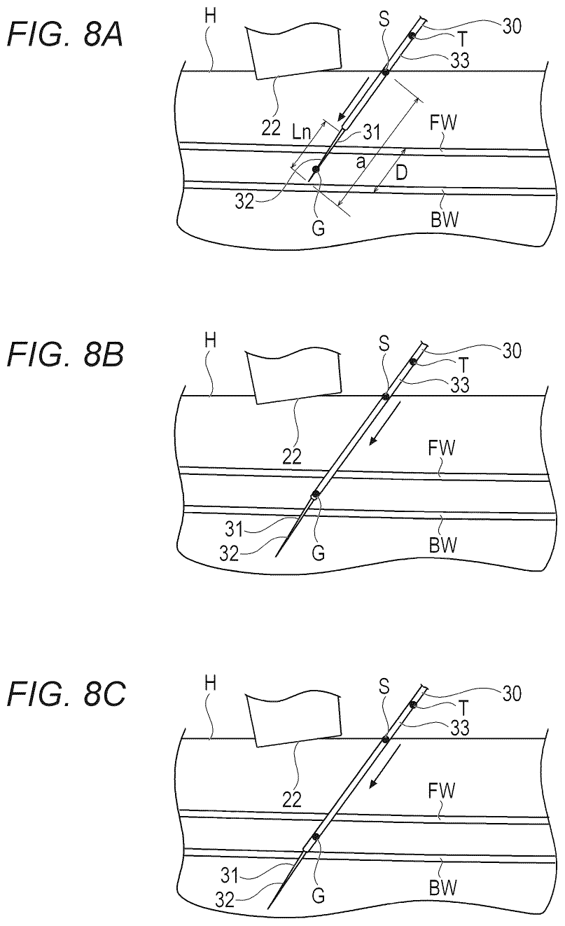

The needle tip 32 is a portion having a blade surface inclined with respect to an axial center on a side closer to the distal end than a portion where the outer diameter of the inner needle 31 is constant. Alternatively, the needle tip 32 may be a portion whose outer diameter decreases toward the most distal end of the needle tip 32 that is sharp. As illustrated in FIG. 8A, a length Ln of the needle tip 32 along the axial center of the inner needle 31 is longer than a diameter D of a target blood vessel to be punctured in a direction along the axial center of the inner needle 31, that is, the length from an adventitial surface of a front wall FW of the blood vessel to an adventitial surface of a back wall BW through the center of gravity of the blood vessel. The front wall FW is a portion through which the puncture unit 30 passes before reaching a lumen of the blood vessel when puncturing the blood vessel. The back wall BW is a portion hit by the puncture unit 30 that has passed through the front wall FW and the lumen of the blood vessel when puncturing the blood vessel. As a result, the needle tip 32 can be made longer than the diameter D of the blood vessel and be sharpened.

As illustrated in FIGS. 1 and 5, the needle tip 32 protrudes from the outer tube 33 in a state where the outer tube 33 covers the outer side of the inner needle 31. An inner needle hub 34 is fixed to a proximal end portion of the inner needle 31. A tubular outer tube hub 35 is fixed to a proximal end portion of the outer tube 33.

As illustrated in FIGS. 1 and 2, the drive unit 40 includes: a first holding portion 41 that holds the inner needle hub 34; a first linear movement portion 42 that linearly moves the first holding portion 41; a second holding portion 47 that holds the outer tube hub 35; a second linear movement portion 48 that linearly moves the second holding portion 47; an inclination portion 43 that inclines the first holding portion 41 and the second holding portion 47; a third linear movement portion 45 that moves the inclination portion 43 in a length direction of the probe 20; and a rotation portion 46 that rotates the third linear movement portion 45 about a predetermined rotation axis P.

The first holding portion 41 can detachably hold the inner needle hub 34. The first holding portion 41 can be, for example, a clamp that can perform holding so as to sandwich the inner needle hub 34.

The first linear movement portion 42 can linearly move the first holding portion 41 holding the inner needle hub 34 of the puncture unit 30 forward and backward along an extending direction (puncture direction) of the inner needle 31. The first linear movement portion 42 is used to adjust a position of the inner needle 31 and puncture the blood vessel with the inner needle 31. The first linear movement portion 42 can include, for example, a rotational drive source such as a motor whose driving can be controlled by the control unit 60, and a structure (for example, a feed screw mechanism) that converts a rotational motion of the rotational drive source into a linear motion.

The second holding portion 47 can detachably hold the outer tube hub 35. The second holding portion 47 can be, for example, a clamp that can perform holding so as to sandwich the outer tube hub 35.

The second linear movement portion 48 can linearly move the second holding portion 47 holding the outer tube hub 35 of the puncture unit 30 forward and backward along an extending direction (puncture direction) of the outer tube 33. The second linear movement portion 48 can adjust a position of the outer tube and push the outer tube 33 into the puncture hole formed by the inner needle 31. The second linear movement portion 48 can include, for example, a rotational drive source such as a motor whose driving can be controlled by the control unit 60, and a structure (for example, a feed screw mechanism) that converts a rotational motion of the rotational drive source into a linear motion.

The inclination portion 43 can incline the first linear movement portion 42 and the second linear movement portion 48. The inclination portion 43 is used to change puncture angles of the inner needle 31 and the outer tube 33 with respect to a surface of a skin of the patient. The inclination portion 43 includes a hinge 44 whose angle can be changed, and a rotational drive source such as a motor whose driving can be controlled by the control unit 60 in order to change the angle of the hinge 44.

The third linear movement portion 45 is used to bring the puncture unit 30 close to (or towards) or away from the skin of the patient. The third linear movement portion 45 can linearly move the inclination portion 43 forward and backward along an extending direction of the probe 20. The third linear movement portion 45 can include, for example, a rotational drive source such as a motor whose driving can be controlled by the control unit 60, and a structure (for example, a feed screw mechanism) that converts a rotational motion of the rotational drive source into a linear motion.

The rotation portion 46 is used to change a direction of the inner needle 31 when the third linear movement portion 45 is viewed substantially perpendicularly to the surface of the skin of the patient. The rotation portion 46 can rotate the inclination portion 43 about the rotation axis P parallel to the length direction of the probe 20. The rotation portion 46 can include, for example, a rotational drive source such as a motor whose driving can be controlled by the control unit 60.

The detection unit 80 can include a first force sensor 81 that detects a force in the puncture direction acting on the inner needle 31 and a second force sensor 82 that detects a force in the puncture direction acting on the outer tube 33. The first force sensor 81 can be disposed, for example, in the first holding portion 41, but a place where the first force sensor 81 is disposed is not limited as long as the force can be detected. The second force sensor 82 can be disposed, for example, in the second holding portion 47, but a place where the second force sensor 82 is disposed is not limited as long as the force can be detected. The first force sensor 81 and the second force sensor 82 transmit detection signals to the control unit 60.

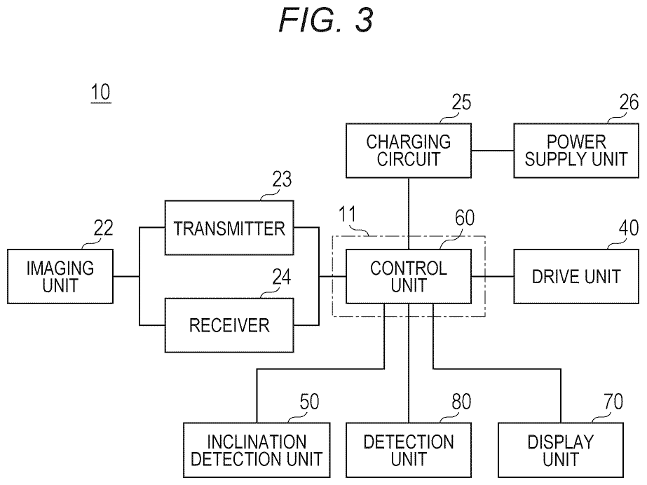

As illustrated in FIGS. 1 and 3, the control unit 60 transmits a signal to the imaging unit 22 via the transmitter 23 and causes the imaging unit 22 to output an ultrasound wave. In addition, the control unit 60 can form a cross-sectional image from a signal obtained from the imaging unit 22 via the receiver 24. Further, the control unit 60 can cause the display unit 70 to display the obtained cross-sectional image. Further, the control unit 60 can perform arithmetic processing such as image analysis from information on the cross-sectional image to control the operation of the drive unit 40. The control unit 60 can include, as physical configurations, a storage circuit and an arithmetic circuit. The storage circuit can store programs and various parameters. The arithmetic circuit can perform arithmetic processing.

The control unit 60 is connected to a power supply unit 26 including a rechargeable battery via a charging circuit 25. In addition, the control unit 60 is connected to the inclination detection unit 50. The control unit 60 may be disposed in the probe 20 or the drive unit 40, or may be configured separately from the probe 20 or the drive unit 40.

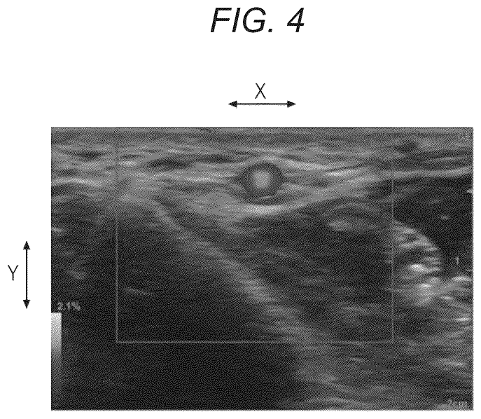

The control unit 60 acquires a cross-sectional image as illustrated in FIG. 4 from the imaging unit 22. It is assumed that a lateral direction in the cross-sectional image, that is, a width direction of the arm H is an X direction, a longitudinal direction in the cross-sectional image, that is, a depth direction of the arm H is a Y direction, and a direction orthogonal to the paper surface of the cross-sectional image, that is, the length direction of the arm H is a Z direction. Coordinates of an upper left point in the cross-sectional image are set as a start point (0, 0, 0).

The control unit 60 can identify a position of the blood vessel in the image by performing image analysis of the acquired cross-sectional image. The control unit 60 also receives the detection signals from the first force sensor 81 and the second force sensor 82. Further, the control unit 60 can control the operation of the drive unit 40. The analysis and control in the control unit 60 will be described in detail later.

As illustrated in FIGS. 3 and 4, the display unit 70 is a monitor or the like capable of displaying the cross-sectional image.

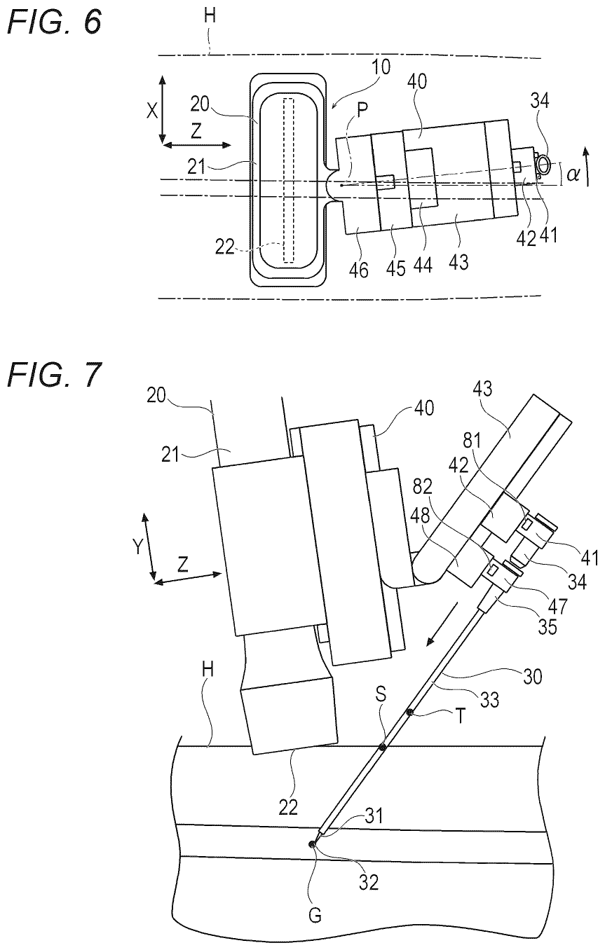

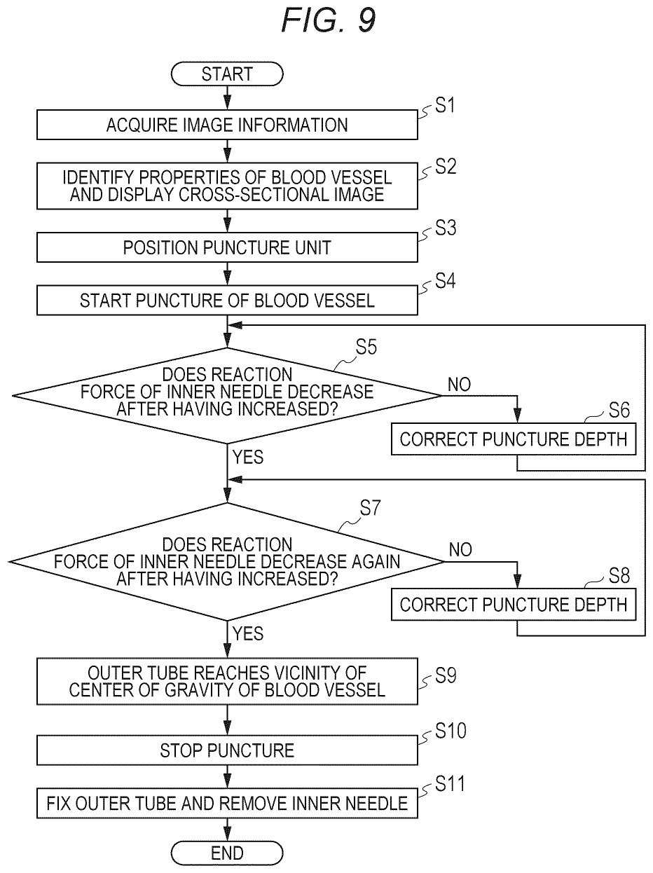

Next, a method of puncturing a blood vessel using the vascular puncture system 10 will be described with reference to a flowchart of the control unit 60 illustrated in FIG. 9. As illustrated in FIGS. 1 and 2, the vascular puncture system 10 is used in contact with the skin surface as illustrated in FIGS. 5 and 6. The solid inner needle 31 in which the length Ln of the needle tip 32 is longer than the outer diameter D in the puncture direction of the target blood vessel is selected and fixed to the first holding portion 41. In addition, the outer tube 33 covering the inner needle 31 is fixed to the second holding portion 47.

The control unit 60 acquires image information from the imaging unit 22 via the receiver 24 (51). The control unit 60 forms a cross-sectional image from the image information. The control unit 60 identifies a position of the blood vessel, the center of gravity of the blood vessel, the blood vessel wall, and the like in the image by performing image analysis on the obtained cross-sectional image, and causes the display unit 70 to display the cross-sectional image (S2). In order to identify the position of the blood vessel, the center of gravity of the blood vessel, a blood vessel wall, and the like in the image, the control unit 60 can prepare a large number of images of the same type and use a technique such as machine learning or deep learning. In addition, it is also possible to detect a region with blood flow by the Doppler method in the imaging unit 22 and recognize the region as a region of the blood vessel.

The control unit 60 sets a position G of the center of gravity of the identified region recognized as the blood vessel in the image as the position of the blood vessel. Coordinates of the detected position G of the center of gravity of the blood vessel are defined as (x, y, 0). Next, the control unit 60 calculates a position (coordinates) and a posture (angle) of the puncture unit 30 desired for puncture, and positions the puncture unit 30 so as to be at the position with the posture (S3). In the present embodiment, the control unit 60 calculates, for example, a preparation position T, a puncture angle θ, and a rotation angle α. The preparation position T is a position of the needle tip 32 immediately before puncture. The puncture angle θ is an angle at which the inner needle 31 at the time of puncture is inclined with respect to a perpendicular line of the skin surface. The rotation angle α is an angle at which the inner needle 31 at the time of puncture is inclined with respect to the Z direction when the surface of the arm H is viewed from a direction of the perpendicular line. The puncture angle θ can also be, for example, a preset angle (for example, 30 degrees). The rotation angle α is set within a range in which the needle tip 32 of the inner needle 31 can reach the inside of an artery. The preparation position T is set at a certain height from the skin surface. The preparation position T is a position where the inner needle 31 can reach the inside of the blood vessel on the cross-sectional image by being caused to protrude along the extending direction (puncture direction).

The control unit 60 first acquires a cross-sectional image from the imaging unit 22. In the cross-sectional image, the Y direction is inclined at the angle of φ with respect to the perpendicular line of the skin surface. In addition, the control unit 60 acquires the inclination φ of the vascular puncture system 10 by the inclination detection unit 50. The control unit 60 sets an upper left end position of the acquired cross-sectional image as a start point (0, 0, 0). With this start point as a reference, the control unit 60 detects the position G of the center of gravity of each blood vessel from the cross-sectional image.

For example, coordinates of the position G of the center of gravity of one detected blood vessel are set to (x, y, 0), and the rotation angle α is set to 0 degrees. A coordinate y1 in the Y direction of a puncture position S on the skin surface can be calculated by y1=y−a·cos(φ+θ) as illustrated in FIG. 5. A coordinate z1 in the Z direction of the puncture position S can be calculated by z1=a·sin(φ+θ). In addition, a puncture depth a is calculated by a=y·cos φ/cos θ. As a result, coordinates (x, y1, z1) of the puncture position S and the puncture depth a are defined.

A distance L from the preparation position T where the needle tip 32 is disposed to the position G of the center of gravity is set to a value longer than the puncture depth a. An angle β between a plane of the cross-sectional image and the puncture direction is obtained by β=θ+φ, and coordinates of the preparation position T can be identified by defining a puncture distance L from the position G of the center of gravity to the puncture position S, the rotation angle α, and the angle β. When the coordinates of the preparation position T are (x, y2, z2) and the rotation angle α is 0 degrees, the coordinate y2 in the Y direction can be calculated by y2=y−L·cos(φ+θ). The coordinate z2 in the Z direction can be calculated by z2=L·sin(φ+θ).

Next, the control unit 60 controls and drives at least one of the first linear movement portion 42, the second linear movement portion 48, the third linear movement portion 45, the inclination portion 43, or the rotation portion 46 such that the inner needle 31 satisfies the puncture distance L, the rotation angle α, and the angle β. As a result, the puncture unit 30 is positioned at the desired position with the desired posture. At this time, the distal end of the needle tip 32 of the inner needle 31 is disposed at the preparation position T. In a case where a relative positional relationship between the inner needle 31 and the outer tube 33 is maintained, the first linear movement portion 42 and the second linear movement portion 48 synchronously move in the same direction by the same length.

Next, the control unit 60 starts puncture of the identified blood vessel (S4). The control unit 60 receives an instruction to start the puncture from the operator by an input means such as a switch, a keyboard, or a mouse connected to the control unit 60. In response to this instruction, the control unit 60 drives the first linear movement portion 42. As a result, the needle tip 32 reaches the blood vessel from the preparation position T through the puncture position S as illustrated in FIG. 7. As illustrated in FIG. 8A, a reaction force received by the inner needle 31 detected by the first force sensor 81 increases when the needle tip 32 punctures the front wall FW of the blood vessel. Therefore, the control unit 60 can detect that the needle tip 32 has punctured and penetrated the front wall FW of the blood vessel by monitoring a temporal change in the reaction force received from the first force sensor 81 and detecting the increase in the reaction force. A punctured state means a state where a part of the needle tip 32 is stuck into a puncture target, and it is unnecessary to stick the entire needle tip 32. Since the reaction force received by the inner needle 31 may increase in the process of passing through the skin and other tissues, the control unit 60 uses a change in the reaction force at the time of reaching the vicinity of the puncture distance L calculated in advance for determination. Alternatively, the control unit 60 may determine that the needle tip 32 has penetrated the front wall FW when the reaction force decreases since the reaction force decreases when the needle tip 32 penetrates the front wall FW after the reaction force increases at the time of puncture. The control unit 60 determines whether the needle tip 32 has punctured and penetrated the front wall FW (S5), and continues the puncture while correcting the assumed puncture depth a as necessary until the needle tip 32 punctures and penetrates the front wall FW (S6).

As illustrated in FIG. 8B, the reaction force received by the inner needle 31 detected by the first force sensor 81 increases again when the needle tip 32 punctures the back wall BW of the blood vessel. Therefore, when determining that the needle tip 32 has punctured and penetrated the front wall FW, the control unit 60 continues to monitor the reaction force received by the inner needle 31 and detected by the first force sensor 81. Then, the control unit 60 can detect that the needle tip 32 starts puncture of the back wall BW of the blood vessel by detecting an increase in the reaction force again. The control unit 60 may determine that the needle tip 32 has penetrated the back wall BW if the reaction force decreases since the reaction force decreases when the needle tip 32 penetrates the back wall BW after the reaction force increased at the time of puncture of the back wall BW. The control unit 60 determines whether the needle tip 32 has punctured and penetrated the back wall BW (S7), and continues the puncture while correcting the assumed puncture depth a as necessary (S8) until the needle tip 32 penetrates the back wall BW.

When detecting the increase in the reaction force received by the inner needle 31 again and determining that the needle tip 32 has punctured and penetrated the back wall BW, the control unit 60 determines that the outer tube 33 has reached the vicinity of the center of gravity of the blood vessel (S9). Then, the control unit 60 stops the movement of the first linear movement portion 42 and the second linear movement portion 48 (S10). As a result, the puncture with the inner needle 31 is completed.

Next, in a state where the second linear movement portion 48 that moves the outer tube 33 is stopped, the control unit 60 drives the first linear movement portion 42 that moves the inner needle 31 to remove the inner needle 31 from the outer tube 33 (S11). Note that either holding of the outer tube 33 or removal of the inner needle 31 may be manually performed. As a result, the control by the control unit 60 is completed.

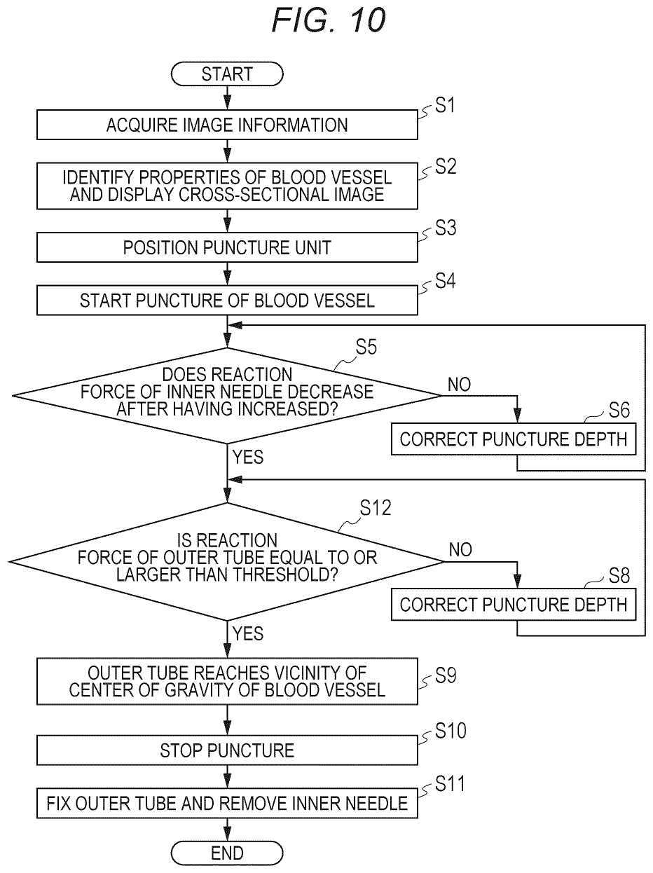

A method of detecting that the distal end of the outer tube 33 has been sufficiently inserted into the blood vessel in the control of the control unit 60 is not limited to the above-described example. For example, as in a first modification illustrated in FIG. 10, the control unit 60 can detect that the needle tip 32 has punctured the back wall BW and the distal end of the outer tube 33 has been sufficiently inserted into the blood vessel by the second force sensor 82 after S5 of determining that the needle tip 32 has punctured the front wall FW based on a measurement result obtained by the first force sensor 81 similarly to the above-described method. The control unit 60 monitors a reaction force received by the outer tube 33 and detected by the second force sensor 82 after the needle tip 32 of the inner needle 31 has punctured the front wall FW. Then, in a case where the reaction force received by the outer tube 33 is equal to or larger than a predetermined threshold or exceeds the threshold, the control unit 60 can detect that the distal end of the outer tube 33 hits an inner wall surface of the back wall BW of the blood vessel as illustrated in FIG. 8C. in a case where the distal end of the outer tube 33 hits the inner wall surface of the back wall BW, the needle tip 32 inevitably punctures the back wall BW. Therefore, the control unit 60 can also detect that the needle tip 32 has punctured the back wall BW by monitoring the reaction force received by the outer tube 33 and detected by the second force sensor 82. The control unit 60 may stop the puncture by detecting an increase in the reaction force at the time of puncture of the back wall BW of the needle tip 32 using the first force sensor 81 and then detecting an increase in the reaction force when the distal end of the outer tube 33 hits the back wall BW using the second force sensor 82.

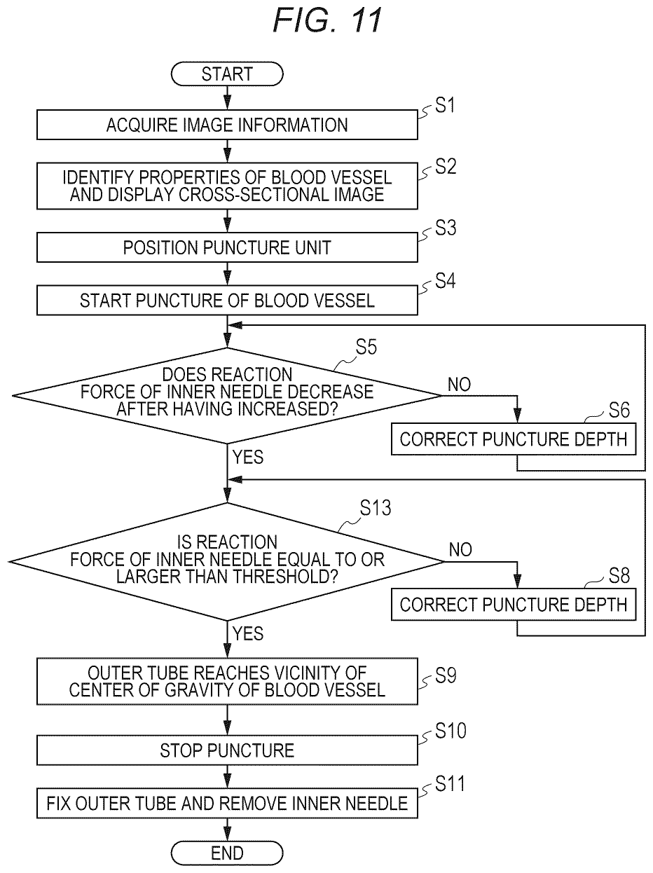

In addition, as in a second modification illustrated in FIG. 11, the control unit 60 may stop the puncture (S13) by detecting contact instead of detecting that the needle tip 32 punctures and penetrates the back wall BW after S5 of determining that the needle tip 32 has punctured the front wall FW based on the measurement result obtained by the first force sensor 81 similarly to the above-described method. The control unit 60 monitors a reaction force detected by the first force sensor 81 after the needle tip 32 of the inner needle 31 has punctured the front wall FW. Then, when the reaction force received by the inner needle 31 is equal to or larger than a predetermined threshold or exceeds the threshold, the control unit 60 can detect that the inner needle 31 has contacted on the inner wall surface of the back wall BW. In a state where the inner needle 31 contacts on the inner wall surface of the back wall BW, the inner needle 31 is not stuck or hardly stuck on the inner wall surface of the back wall BW. The threshold for detecting the contact of the inner needle 31 on the inner wall surface of the back wall BW is smaller than a threshold for detecting that the inner needle 31 has punctured and penetrated the back wall BW. The control unit 60 detects that the distal end of the outer tube 33 is sufficiently inserted into the blood vessel by detecting that the distal end of the inner needle 31 contacts on the inner wall surface of the back wall BW, and stops the movement of the first linear movement portion 42 and the second linear movement portion 48.

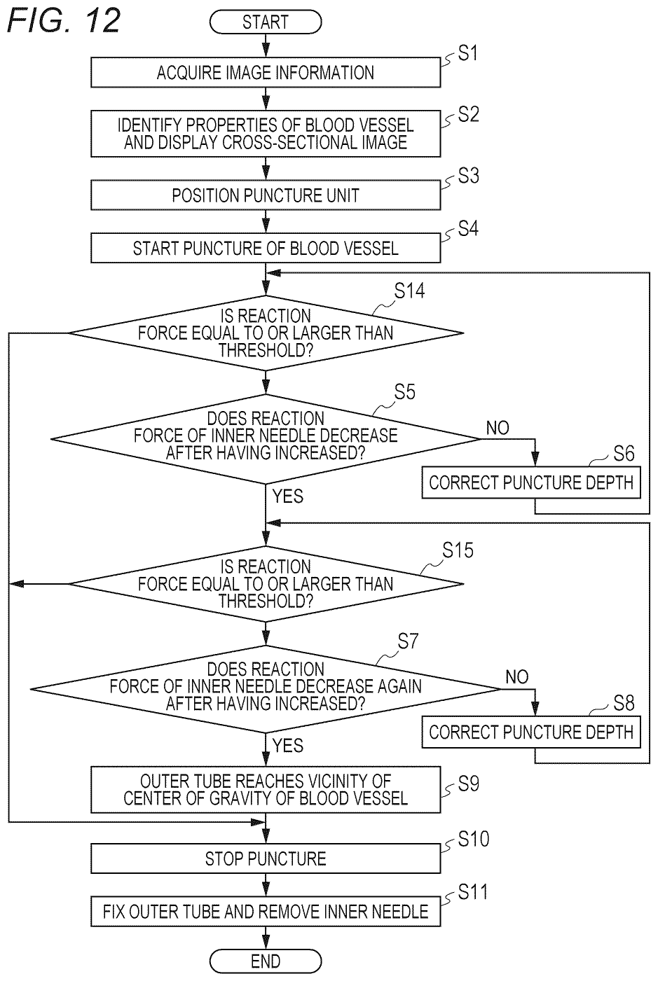

In addition, as in a third modification illustrated in FIG. 12, before the inner needle 31 punctures and penetrates the front wall FW and before the inner needle 31 punctures and penetrates the back wall BW, the control unit 60 may determine whether a measurement result obtained by the first force sensor 81 is equal to or larger than a predetermined threshold or exceeds the threshold (S14 and S15). In a case where the measurement result obtained by the first force sensor 81 is equal to or larger than the predetermined threshold or exceeds the threshold, it is determined that the needle tip 32 hits, for example, a radius, and the movement of the first linear movement portion 42 and the second linear movement portion 48 is stopped. As a result, the safety of the vascular puncture system 10 can be enhanced.

After removing the inner needle 31 with the outer tube 33 being left, the operator inserts a guide wire from a proximal end opening of the outer tube hub 35 by a defined length. Subsequently, the operator removes the outer tube 33 with the guide wire being left, whereby a procedure for securing an access route to the blood vessel is completed.

As described above, the vascular puncture device 11 according to the present embodiment is the vascular puncture device 11 that punctures a blood vessel using the imaging unit 22 that comes into contact with a skin surface and acquires a cross-sectional image of a human body, the inner needle 31 including the needle tip 32 that is sharp, the outer tube 33 that is flexible and covers the inner needle 31, the drive unit 40 that moves the inner needle 31 and the outer tube 33, and the detection unit 80 (the first force sensor 81 and/or the second force sensor 82) that detects entry of a distal end of the outer tube 33 into the blood vessel or puncture of the needle tip 32 into the back wall BW of the blood vessel, and includes the control unit 60 that is capable of receiving information on the cross-sectional image, controlling an operation of the drive unit 40, and receiving a detection result from the detection unit 80. The control unit 60 controls the drive unit 40 to move the inner needle 31 and the outer tube 33 and cause the inner needle 31 to puncture the blood vessel, and controls the drive unit 40 to stop the puncture with the inner needle 31 in a case where the detection unit 80 detects the entry of the distal end of the outer tube 33 into the blood vessel or detects contact or puncture of the needle tip 32 with respect to the back wall BW of the blood vessel.

The vascular puncture device 11 configured as described above can appropriately dispose the distal end of the outer tube 33 in the blood vessel regardless of skill of an operator when automatically performing vascular puncture using the inner needle 31 by stopping the puncture in a case where the entry of the distal end of the outer tube 33 into the blood vessel is detected or stopping the puncture in a case where the contact or puncture of the needle tip 32 with respect to the back wall BW of the blood vessel is detected.

In addition, the control unit 60 receives a detection result from the first force sensor 81 that is provided in the detection unit 80 and detects a reaction force acting on the inner needle 31 at the time of puncture. In a case where the reaction force detected by the first force sensor 81 increases and then increases again, the control unit 60 determines that the needle tip 32 has punctured the back wall BW and stops the puncture with the inner needle 31. As a result, the vascular puncture device 11 can detect that the inner needle 31 has punctured the front wall FW and then the back wall BW of the blood vessel by the first force sensor 81 without requiring the operator to confirm backflow of blood in the inner needle 31. Therefore, the vascular puncture device 11 can appropriately secure an access route by the outer tube 33 and can reduce bleeding. In addition, the vascular puncture device 11 can simplify a procedure without requiring an operation of suppressing bleeding.

In addition, the control unit 60 may receive a detection result from at least one force sensor (in the present embodiment, the first force sensor 81 and the second force sensor 82) that is provided in the detection unit 80 and detects a reaction force acting on the inner needle 31 and a reaction force acting on the outer tube 33 at the time of puncture. The control unit 60 may determine that the outer tube 33 hits the back wall BW after the puncture of the back wall BW by the needle tip 32 and stop the puncture with the inner needle 31 in a case where the reaction force acting on the outer tube 33 detected by the second force sensor 82 increases after the reaction force acting on the inner needle 31 detected by the first force sensor 81 increases. As a result, the vascular puncture device 11 can detect that the distal end of the outer tube 33 has entered the blood vessel until the distal end hits the inner wall surface of the back wall BW of the blood vessel by the second force sensor 82 without requiring the operator to confirm the backflow of blood in the inner needle 31. Therefore, the vascular puncture device 11 can appropriately secure an access route by the outer tube 33 and can reduce bleeding. In addition, the vascular puncture device 11 can simplify a procedure without requiring an operation of suppressing bleeding.

In addition, the control unit 60 stops the puncture with the inner needle 31 in a case where the reaction force of the inner needle 31 detected by the first force sensor 81 is equal to or larger than a threshold or exceeds the threshold. As a result, the vascular puncture device 11 can detect that the inner needle 31 hits, for example, a bone other than the blood vessel and stop the puncture. Therefore, the safety of the vascular puncture device 11 can be improved.

In addition, the control unit 60 may detect the entry of the distal end of the outer tube 33 into the blood vessel by the detection unit 80 or detect the contact or puncture of the needle tip 32 with respect to the back wall BW of the blood vessel by a machine-learned model from detection results from the detection unit 80. As a result, the control unit 60 can perform relatively highly accurate detection based on a plurality of pieces of stacked data.

The vascular puncture system 10 according to the present embodiment is the vascular puncture system 10 that includes: the imaging unit 22 that comes into contact with a skin surface and acquires a cross-sectional image of a human body; the inner needle 31 including the needle tip 32 that is sharp; the outer tube 33 that is flexible and covers the inner needle 31; the drive unit 40 that moves the inner needle 31 and the outer tube 33; a detection unit (the first force sensor 81 and/or the second force sensor 82) that detects entry of a distal end of the outer tube 33 into a blood vessel or puncture of the needle tip 32 into the back wall BW of the blood vessel; and the control unit 60 that is capable of receiving information on the cross-sectional image and controlling an operation of the drive unit 40. The control unit 60 controls the drive unit 40 to move the inner needle 31 and the outer tube 33 to puncture the blood vessel with the inner needle 31, and controls the drive unit 40 to stop the puncture with the inner needle 31 in a case where the detection unit detects the entry of the distal end of the outer tube 33 into the blood vessel or detects puncture or contact of the needle tip 32 with respect to the back wall BW of the blood vessel.

The vascular puncture system 10 configured as described above can appropriately dispose the distal end of the outer tube 33 in the blood vessel regardless of skill of an operator when automatically performing vascular puncture using the inner needle 31 by stopping the puncture in a case where the entry of the distal end of the outer tube 33 into the blood vessel is detected or stopping the puncture in a case where the puncture of the needle tip 32 with respect to the back wall BW of the blood vessel is detected.

In addition, the needle tip 32 of the inner needle 31 is longer than the outer diameter D in a puncture direction of a target blood vessel to be punctured. As a result, the needle tip 32 of the inner needle 31 becomes sharp, and thus, it is difficult for the blood vessel to escape when being punctured, and pain can be reduced. Note that a length of the needle tip 32 may be equal to or less than the outer diameter D in the puncture direction of the target blood vessel.

In addition, the inner needle 31 is solid. As a result, the vascular puncture system 10 can reduce an outer diameter of the inner needle 31 while maintaining rigidity, and thus, bleeding can be reduced. In addition, the vascular puncture system 10 can simplify a procedure without requiring an operation of suppressing bleeding. The inner needle 31 may be hollow.

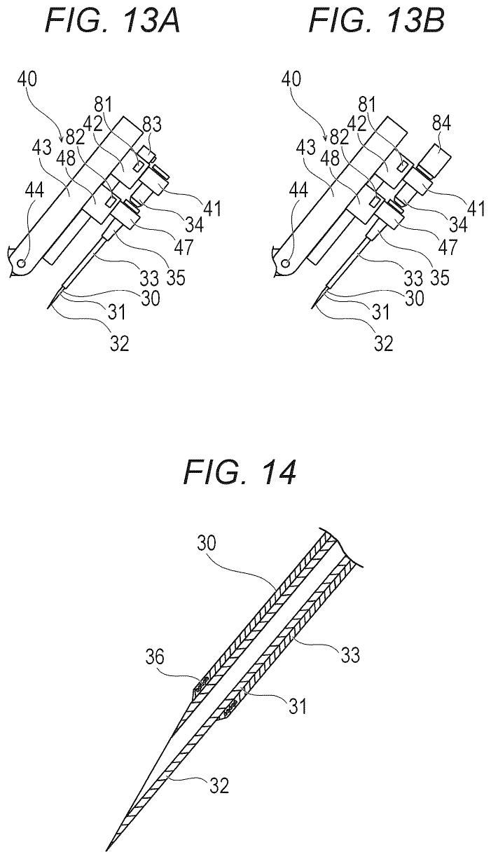

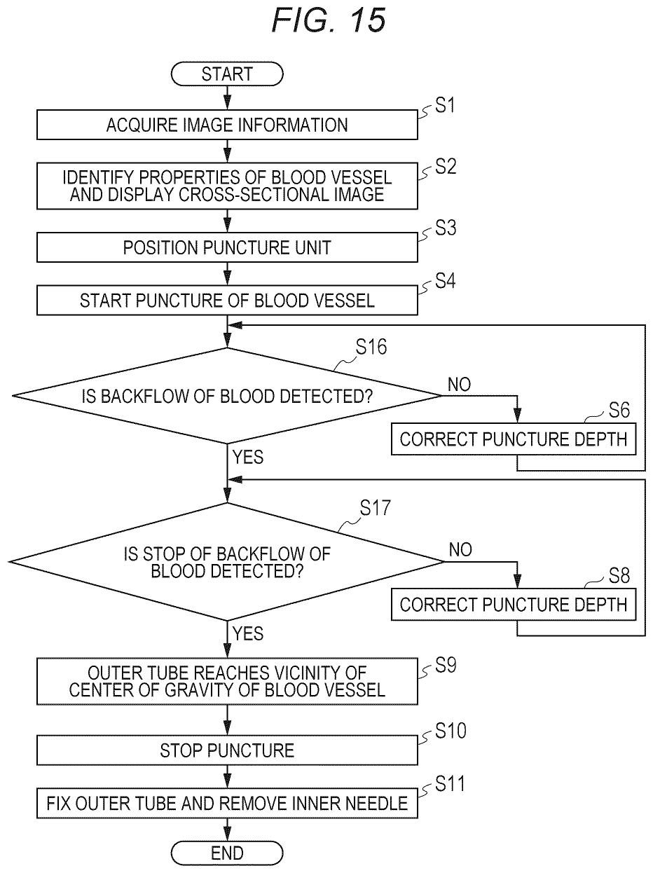

The present disclosure is not limited to the above-described embodiment, and various modifications can be made by those skilled in the art within the technical idea of the present disclosure. For example, as in a fourth modification illustrated in FIGS. 13A and 15, the detection unit may be an optical sensor 83 that detects backflow of blood from the inner needle 31 at the time of puncture, instead of a force sensor. The optical sensor 83 can be, for example, a camera including an imaging element. A structure of the optical sensor 83 is not particularly limited as long as it is possible to optically detect the backflow of blood from the inner needle 31. The optical sensor 83 is disposed at a position where the inner needle hub 34 can be observed (for example, the first linear movement portion 42 and the first holding portion 41). The optical sensor 83 may be fixed to the inner needle hub 34. The control unit 60 can receive a detection result from the optical sensor 83. In a case where the optical sensor 83 detects the occurrence of backflow of blood, the control unit 60 determines that the inner needle 31 has punctured and penetrated the front wall FW (S16). Then, when detecting a stop of the backflow or a decrease in the amount of blood flowing backward after the occurrence of the backflow (S17), the control unit 60 determines that the inner needle 31 has punctured and penetrated the back wall BW, and the puncture with the inner needle 31 is stopped. Therefore, the vascular puncture system 10 can detect the backflow of blood in the inner needle 31, and thus, can dispose the inner needle 31 and the outer tube 33 appropriately with respect to the blood vessel regardless of skill of an operator.

In addition, as in a fifth modification illustrated in FIGS. 13B and 15, the detection unit may be a flow meter 84 that detects backflow of blood from the inner needle 31 at the time of puncture, instead of a force sensor. The flow meter 84 is fixed to an opening of the inner needle hub 34. Alternatively, the flow meter 84 may be fixed to the opening of the inner needle hub 34 via a tube for circulating blood. The control unit 60 can receive a measurement result from the flow meter 84. The control unit 60 determines that the inner needle 31 has punctured and penetrated the front wall FW in a case where the flow meter 84 detects the backflow of blood (S16). Then, the control unit 60 determines that the inner needle 31 has punctured and penetrated the back wall BW and stops the puncture with the inner needle 31 in a case where a stop of the backflow or a decrease in the amount of blood flowing backward after the occurrence of the backflow is detected (S17). Therefore, the vascular puncture system 10 can detect the backflow of blood in the inner needle 31, and thus, can dispose the inner needle 31 and the outer tube 33 appropriately with respect to the blood vessel regardless of skill of an operator. In addition, the force sensor and the optical sensor may be used in combination. For example, the control unit 60 determines that puncture of a blood vessel is successful and stops the puncture when the inner needle 31 punctures the front wall FW and reverse blood is confirmed by the camera, the inner needle 31 contacts on a radius, and pressure applied to the inner needle 31 is equal to or higher than a certain level (or predetermined level). On the other hand, when the pressure applied to the inner needle 31 is equal to or higher than the certain level (or predetermined level) but the reverse blood is not confirmed by the camera, it can be determined that the inner needle 31 has failed in puncturing the blood vessel, and movement of the drive unit 40 is stopped. Thereafter, a cross section of the blood vessel is observed again by the imaging unit 22, which is an ultrasound probe, a puncture position is adjusted again, and the puncture is resumed.

In addition, as in a sixth modification illustrated in FIG. 14, the detection unit may be an ultrasound marker 36 disposed at a distal end portion of the outer tube 33. The ultrasound marker 36 is made of a material that can be visually observed in an obtained cross-sectional image. The material of the ultrasound marker 36 is a material having an acoustic impedance higher or lower than that of moisture or tissue in a body, and can be, for example, stainless steel. The ultrasound marker 36 can be, for example, a coil embedded in the distal end portion of the outer tube 33, but a structure of the ultrasound marker 36 is not limited. Therefore, the ultrasound marker 36 is not necessarily embedded in the outer tube 33, or may be a tubular body, a ring, or the like instead of the coil, or may have an uneven shape disposed on the surface of the outer tube. The control unit 60 can identify a position of the ultrasound marker 36 from the obtained cross-sectional image. Therefore, the control unit 60 monitors the ultrasound marker 36, and stops the first linear movement portion 42 and the second linear movement portion 48 when the distal end portion of the outer tube 33 in which the ultrasound marker 36 is disposed reaches an appropriate position inside a blood vessel. As a result, puncture with the inner needle 31 can be stopped, and the outer tube 33 can be appropriately disposed with respect to the blood vessel. Therefore, the vascular puncture system 10 can detect that the distal end of the outer tube 33 is disposed at a desired position of the blood vessel by the ultrasound marker 36 without requiring an operator to confirm backflow of blood in the inner needle 31.

The drive unit 40 can have five movable portions (the first linear movement portion 42, the second linear movement portion 48, the third linear movement portion 45, the rotation portion 46, and the inclination portion 43), but the number of movable portions may be six or more or four or less.

In addition, the position G of the center of gravity of the blood vessel to be punctured is detected from the cross-sectional image, and the puncture position S on the skin surface and the preparation position T are calculated from the position G of the center of gravity in the present embodiment. However, the puncture position S and the preparation position T may be calculated by detecting a position other than the position G of the center of gravity of a blood vessel to be punctured. For example, the control unit 60 may detect a position in an inner surface of a blood vessel to be punctured located between the blood vessel and the imaging unit 22 or in a membrane of the blood vessel from a cross-sectional image, and calculate the puncture position S and the preparation position T based on coordinates of the position. In addition, the control unit 60 may detect the position in the inner surface of the blood vessel to be punctured located between the blood vessel and the imaging unit 22 or in the membrane of the blood vessel from the cross-sectional image, and calculate the puncture position S and the preparation position T from coordinates of a position separated from this position by a certain distance (or predetermined distance).

In addition, the drive unit 40 may be a robot arm.

In addition, the vascular puncture device 11 or the vascular puncture system 10 may have a function of displaying a blood vessel determined to be punctured or a medical device adapted to a punctured blood vessel. An operator can insert, for example, a sheath along the outer tube 33 after puncturing the blood vessel with the puncture unit 30 and removing the inner needle 31. An outer diameter of the sheath is preferably equal to or smaller than an inner diameter of the blood vessel into which the sheath is to be inserted. This is because when the outer diameter of the sheath is equal to or larger than the inner diameter of the blood vessel, complications are likely to be caused by inserting the sheath into the blood vessel. As an example of a method of calculating the inner diameter of the blood vessel, a length of a diagonal line passing through a center of gravity of an inner peripheral surface of the identified blood vessel (artery or vein) is acquired for the entire circumference at predetermined angle intervals (for example, in the interval of 1 degree), and an average value of the length of a diagonal line passing through a center of gravity of an inner peripheral surface of the identified blood vessel (artery or vein) can be set as the inner diameter of the blood vessel. Alternatively, the inner diameter of the blood vessel may be calculated back from an area of the inside of the inner peripheral surface of the blood vessel. When an inner diameter of a blood vessel of an artery, it is preferable to detect the inner diameter of the blood vessel at a certain timing since the artery pulsates. In addition, the certain timing is preferably a timing when the blood vessel contracts most. Since the minimum inner diameter of the inner diameter of the blood vessel is larger than the outer diameter of the medical device to be inserted, the occurrence of complications can be reduced. After calculating the inner diameter of the blood vessel, the control unit 60 can display the outer diameter and a product type of the medical device adapted to the calculated inner diameter of the blood vessel on a display device such as a monitor together with the cross-sectional image. The control unit 60 may identify at least one of an optimal outer diameter, length, or product type of the inner needle 31 from information on a blood vessel determined to be punctured, past statistical information, and the like, display the same on a display device such as a monitor together with a cross-sectional image to present the same to the operator.

The detailed description above describes embodiments of a vascular puncture device and a vascular puncture system capable of detecting and puncturing a position of a blood vessel from an image acquired by an echographic device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A vascular puncture device configured to puncture a blood vessel, the vascular puncture device comprising:
an imaging unit configured to come into contact with a skin surface and acquire a cross-sectional image of a human body;
an inner needle including a needle tip;
an outer tube that is flexible and configured to cover the inner needle;
a drive unit configured to move the inner needle and the outer tube;
a detection unit configured to detect entry of a distal end of the outer tube into the blood vessel or contact or puncture of the needle tip with respect to a back wall of the blood vessel;
a control unit configured to receive information on the cross-sectional image, control an operation of the drive unit, and receive a detection result from the detection unit; and
wherein the control unit is configured to:
control the drive unit to move the inner needle and the outer tube to puncture the blood vessel with the inner needle; and
control the drive unit to stop the puncture with the inner needle in a case where the detection unit is configured to detect the entry of the distal end of the outer tube into the blood vessel or detect the contact or puncture of the needle tip with respect to the back wall of the blood vessel.

2. The vascular puncture device according to claim 1, wherein the control unit is configured to:
receive a detection result from a force sensor that is provided in the detection unit and detect a reaction force acting on the inner needle during puncture; and
determine that the needle tip punctures the back wall and stop the puncture with the inner needle in a case where the reaction force detected by the force sensor increases and then increases again.

3. The vascular puncture device according to claim 1, wherein the control unit is configured to:
receive a detection result from at least one force sensor that is provided in the detection unit and detect a reaction force acting on the inner needle and a reaction force acting on the outer tube during puncture; and
determine that the outer tube hits the back wall after the back wall is punctured by the needle tip and stop the puncture with the inner needle in a case where the reaction force acting on the outer tube detected by the force sensor increases after the reaction force acting on the inner needle detected by the force sensor increases.

4. The vascular puncture device according to claim 2, wherein the control unit is configured to stop the puncture with the inner needle in a case where the reaction force on the inner needle detected by the force sensor is equal to or larger than a threshold.

5. The vascular puncture device according to claim 1, wherein the control unit is configured to:
receive a detection result from an optical sensor that is provided in the detection unit and detect backflow of blood from the inner needle during puncture; and
stop the puncture with the inner needle in a case where the optical sensor detects occurrence of the backflow of the blood and stop of the backflow after the occurrence.

6. The vascular puncture device according to claim 1, wherein the control unit is configured to:
receive a detection result from a flow rate sensor that is provided in the detection unit and detect backflow of blood from the inner needle during puncture; and
stop the puncture with the inner needle in a case where the flow rate sensor detects occurrence of the backflow of the blood and stop of the backflow after the occurrence.

7. The vascular puncture device according to claim 1, wherein the control unit is configured to receive a detection result from an ultrasound marker that is provided in the detection unit and disposed in a distal end portion of the outer tube.

8. The vascular puncture device according to claim 1, wherein the control unit is configured to use a machine-learned model obtained from detection results from the detection unit to detect the entry of the distal end of the outer tube into the blood vessel or detect the contact or puncture of the needle tip with respect to the back wall of the blood vessel by the detection unit.

9. A vascular puncture system comprising:
an imaging unit configured to come into contact with a skin surface and acquire a cross-sectional image of a human body;
an inner needle including a needle tip;
an outer tube that is flexible and configured to cover the inner needle;
a drive unit configured to move the inner needle and the outer tube;
a detection unit configured to detect entry of a distal end of the outer tube into the blood vessel or contact or puncture of the needle tip with respect to a back wall of the blood vessel;
a control unit configured to receive information on the cross-sectional image, control an operation of the drive unit, and receive a detection result from the detection unit; and
wherein the control unit is configured to:
control the drive unit to move the inner needle and the outer tube to puncture the blood vessel with the inner needle; and
control the drive unit to stop the puncture with the inner needle in a case where the detection unit detects the entry of the distal end of the outer tube into the blood vessel or detects the contact or puncture of the needle tip with respect to the back wall of the blood vessel.

10. The vascular puncture system according to claim 9, wherein the needle tip of the inner needle is longer than an outer diameter in a puncture direction of a target blood vessel to be punctured.

11. The vascular puncture system according to claim 9, wherein the inner needle is solid.

12. The vascular puncture system according to claim 9, wherein the control unit is configured to:
receive a detection result from a force sensor that is provided in the detection unit and detect a reaction force acting on the inner needle during puncture; and
determine that the needle tip punctures the back wall and stop the puncture with the inner needle in a case where the reaction force detected by the force sensor increases and then increases again.

13. A method for puncturing a blood vessel, the method comprising:
acquiring a cross-sectional image of a human body;
detecting entry of a distal end of an outer tube into the blood vessel or contact or puncture of a needle tip of an inner needle with respect to a back wall of the blood vessel, the outer tube configured to cover the inner needle;
moving the inner needle and the outer tube to puncture the blood vessel with the inner needle; and
stopping the puncture with the inner needle when in a case where the detection of the entry of the distal end of the outer tube into the blood vessel or the detection of contact or puncture of the needle tip with respect to the back wall of the blood vessel.

14. The method according to claim 13, further comprising:
receiving a detection result from a force sensor and detecting a reaction force acting on the inner needle during puncture; and
determining that the needle tip punctures the back wall and stopping the puncture with the inner needle in a case where the reaction force detected by the force sensor increases and then increases again.

15. The method according to claim 13, further comprising:
receiving a detection result from at least one force sensor and detecting a reaction force acting on the inner needle and a reaction force acting on the outer tube during puncture; and
determining that the outer tube hits the back wall after the back wall is punctured by the needle tip and stopping the puncture with the inner needle in a case where the reaction force acting on the outer tube detected by the force sensor increases after the reaction force acting on the inner needle detected by the force sensor increases.

16. The method according to claim 14, further comprising:
stopping the puncture with the inner needle in a case where the reaction force on the inner needle detected by the force sensor is equal to or larger than a threshold.

17. The method according to claim 13, further comprising:
receiving a detection result from an optical sensor and detecting backflow of blood from the inner needle during puncture; and
stopping the puncture with the inner needle in a case where the optical sensor detects occurrence of the backflow of the blood and stop of the backflow after the occurrence.

18. The method according to claim 13, further comprising:
receiving a detection result from a flow rate sensor and detecting backflow of blood from the inner needle during puncture; and
stopping the puncture with the inner needle in a case where the flow rate sensor detects occurrence of the backflow of the blood and stopping of the backflow after the occurrence.

19. The method according to claim 13, further comprising:
receiving a detection result from an ultrasound marker that is provided in the detection unit and disposed in a distal end portion of the outer tube.

20. The method according to claim 13, further comprising:
detecting the entry of the distal end of the outer tube into the blood vessel using a machine learning model; or
detecting the contact or puncture of the needle tip with respect to the back wall of the blood vessel using the machine learning model.
